(19) United States
(12) Patent Application Publication
(10) Pub. No.: US 2006/0074272 A1
(43) Pub. Date: Apr. 6, 2006

(54) Portable system for assessing urinary function and peforming endometrial ablation

(76) Inventor: Anthony DiUbaldi, Jackson, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/959,264

(22) Filed: Oct. 6, 2004

(51) Int. Cl. A61F 2/00

(52) U.S. Cl. 600/29

(57) ABSTRACT

A medical system is provided that includes a control device and a plurality of modules each capable of being removably coupled to the control device. When a first one of the plurality of modules is removably coupled to the control device, the medical system is capable of performing a test to assess urinary function, and when a second one of the plurality of modules is removably coupled to the control device, the medical system is capable of performing endometrial ablation.

### FIELD OF THE INVENTION

The present invention relates generally to a portable system for both assessing urinary function and for performing endometrial ablation.

### BACKGROUND OF THE INVENTION

Women account for more than 11 million of incontinence cases. Moreover, a majority of women with incontinence suffer from stress urinary incontinence (SUI). Women with SUI involuntarily lose urine during normal daily activities and movements, such as laughing, coughing, sneezing and regular exercise.

SUI may be caused by a functional defect of the tissue or ligaments connecting the vaginal wall with the pelvic muscles and pubic bone. Common causes include repetitive straining of the pelvic muscles, childbirth, loss of pelvic muscle tone and estrogen loss. Such a defect results in an improperly functioning urethra. Unlike other types of incontinence, SUI is not a problem of the bladder.

Normally, the urethra, when properly supported by strong pelvic floor muscles and healthy connective tissue, maintains a tight seal to prevent involuntary loss of urine. When a woman suffers from the most common form of SUI, however, weakened muscle and pelvic tissues are unable to adequately support the urethra in its correct position. As a result, during normal movements when pressure is exerted on the bladder from the diaphragm, the urethra cannot retain its seal, permitting urine to escape. Because SUI is both embarrassing and unpredictable, many women with SUI avoid an active lifestyle, shying away from social situations.

SUI is categorized into three types. Type I and Type II are directed to urethral hypermobility. Type III is directed to intrinsic sphincter deficiency (ISD). Proper treatment of incontinence necessarily requires identification of the cause and type of incontinence, which is accomplished by urodynamic evaluation.

A much simplified system and method for assessing urinary function is described in detail in U.S. patent application Ser. No. 10/183,790, filed on Jun. 27, 2002, and published on Jan. 30, 2003 (Publication No. 2003/0023135), which is incorporated herein by reference in its entirety.

Another medical condition that afflicts millions of women is menorraghia, or heavy uterine bleeding. This condition often has a severe negative impact on a woman's quality of life, causing pain and often interrupting or preventing normal daily routines. Menorraghia is often treated by effecting necrosis of the endometrial lining of the uterus. One device and method for effecting necrosis involves inserting a distendable bladder into the uterus, infusing fluid into the bladder to expand it against the inner lining of the uterus, and subsequently heating the fluid within the bladder to a sufficient temperature and for a sufficient time period to cause necrosis of the endometrial lining of the uterus that is in contact with the bladder. This type of device and method is described in greater detail in U.S. Pat. Nos. 4,949,718, 5,105,808 and 5,704,934, which are incorporated herein by reference in their entirety. Such a device is also currently sold by Gynecare, a division of Ethicon, Inc. of Somerville, N.J., under the name Thermachoice®.

To date, completely different sets of equipment are required to perform endometrial ablation and incontinence testing. It would be desirable to provide a single system that easily and cost effectively enables performance of both incontinence testing and endometrial ablation.

### SUMMARY OF THE INVENTION

The present invention provides a medical system including a control device and a plurality, of modules each capable of being removably coupled to the control device. When a first one of the plurality of modules is removably coupled to the control device, the medical system is capable of performing a test to assess urinary function, and when a second one of the plurality of modules is removably coupled to the control device, the medical system is capable of performing endometrial ablation. The first and second modules may further include a tubing assembly forming a fluid conduit from a fluid inlet to a fluid outlet, and the control device may further include a pump device that couples with the module tubing assemblies for pumping fluid therethrough when one of the modules is coupled to the control device.

In one embodiment, the second module includes an endometrial ablation system including a catheter having a proximal end and a distal end, a distendable bladder attached to the proximal end for insertion into a patient's uterus, and a heater for heating fluid infused into the distendable bladder. The fluid inlet of the second module is capable of coupling with a fluid source and the fluid outlet is in fluid communication with an interior of the distendable bladder, and when the second module tubing assembly is coupled with the pump, operation of the pump controls the flow of fluid from the fluid source into or out of the distendable bladder.

In yet another embodiment, the control device further includes a pressure sensor and the second module further includes a pressure interface that, when the second module is coupled with the control device, is positioned relative to the control device pressure sensor so as to transmit pressure information thereto. The pressure interface may be in fluid communication with the tubing assembly of the second module at a position such that fluid pressure at the pressure interface substantially corresponds to pressure within the distendable bladder. The second module in yet another embodiment may include at least one temperature sensing element for sensing a temperature of fluid within the distendable bladder, and temperature from the at least one temperature sensing element may be provided to the control unit through an electrical interface, with the heater being controlled by the control unit via the electrical interface. In yet another embodiment, the second module further includes an external power connector for coupling with a power source.

In another embodiment, the when the first module is removably coupled to the control device, the system measures Urethral Resistance Pressure to thereby assess urinary function. The first module may also further include an insert member dimensioned for at least partial insertion into a patient's urinary tract and coupled to the first module fluid outlet so that fluid infused through the first module tubing assembly passes through the insert member and into the urinary tract. The insert member may be dimensioned for insertion into the urethral canal distal of the urethral sphincter, and the first module may further include a pressure interface in fluid communication with the urethral canal distal of the urethral sphincter when the insert member is so inserted.

The present invention also provides a medical system including a control device including at least one input device, at least one output device, a microprocessor, and a pump device, and first and second modules each individually capable of being removably coupled with the control device. When the first module is so coupled the system is capable of performing a test to assess urinary function, and when the second module is so coupled the system is capable of performing endometrial ablation. The first and second modules each include a tubing assembly extending from a fluid inlet to a fluid outlet, wherein when coupled to the control device, the tubing assembly is coupled to the pump device for pumping fluid therethrough. The first module further includes a device coupled thereto for infusing fluid into a patient's urinary tract, wherein feedback is provided regarding the infused fluid to the control device and the infusion of fluid into the patient's urinary tract is controlled by the control unit. The second module further includes an ablation system coupled thereto for ablating an interior lining of the uterus, wherein feedback is provided from the ablation system to the control device and wherein the control device controls operation of the ablation system.

These and other features and advantages of the present invention will become apparent from the following more detailed description, when taken in conjunction with the accompanying drawings which illustrate, by way of example, the principles of the invention.

### BRIEF DESCRIPTION OF THE DRAWINGS

FIG. 1 is a perspective view of a one embodiment of a portable medical system according to the present invention;

FIG. 2 is a front perspective view of a control device according to the present invention;

FIG. 3 is a rear perspective view of the control device of FIG. 2;

FIG. 4 is an exploded perspective view illustrating interaction of a control device identification mechanism and module identification components;

FIG. 5a is a schematic cross-sectional view taken across line 5a-5a of FIG. 5 prior to engagement of the control device with the test module;

FIG. 5b is a schematic cross-sectional view similar to FIG. 5a showing engagement of the control device with the test module;

FIG. 6 is a front perspective view of a module according to the present invention;

FIG. 7 is a schematic illustration of one embodiment of control device electronics assembly;

FIGS. 8a-8f are flow diagrams illustrating operation of control device software and graphical user interface components;

FIG. 9 is an alternate embodiment of a medical system according to the present disclosure;

FIG. 10 is a schematic representation of a portable medical system including an SUI module;

FIG. 10a is a partial cross-sectional view of one embodiment of a portable medical system including an SUI module;

FIG. 10b is an enlarged illustration of a pressure transducer and pressure interface;

FIG. 11a is a side elevational view and partial cross-section of one embodiment of a hand actuator in an assembled configuration;

FIG. 11b is a side elevational view and partial cross-section of the hand actuator of FIG. 11a in an unassembled configuration;

FIG. 11c is a side elevational view and partial cross-section of the hand actuator of FIG. 11a in an operational mode;

FIG. 11d is an alternative embodiment of a hand actuator according to the present invention;

FIG. 12 is an enlarged perspective view of one embodiment of a meatus plug device;

FIG. 13 is a schematic view of illustrating one embodiment of a urodynamic system in relation to a female urinary/reproductive system;

FIG. 14 is a flow diagram illustrating steps for using the system of FIG. 10;

FIG. 15 is an exploded perspective view of a endometrial ablation module in conjunction with a control device;

FIGS. 16a and 16b illustrate an embodiment of distendable bladder within a uterus; and

FIG. 17 illustrates one embodiment of the circuitry present within the endometrial ablation module.

### DETAILED DESCRIPTION

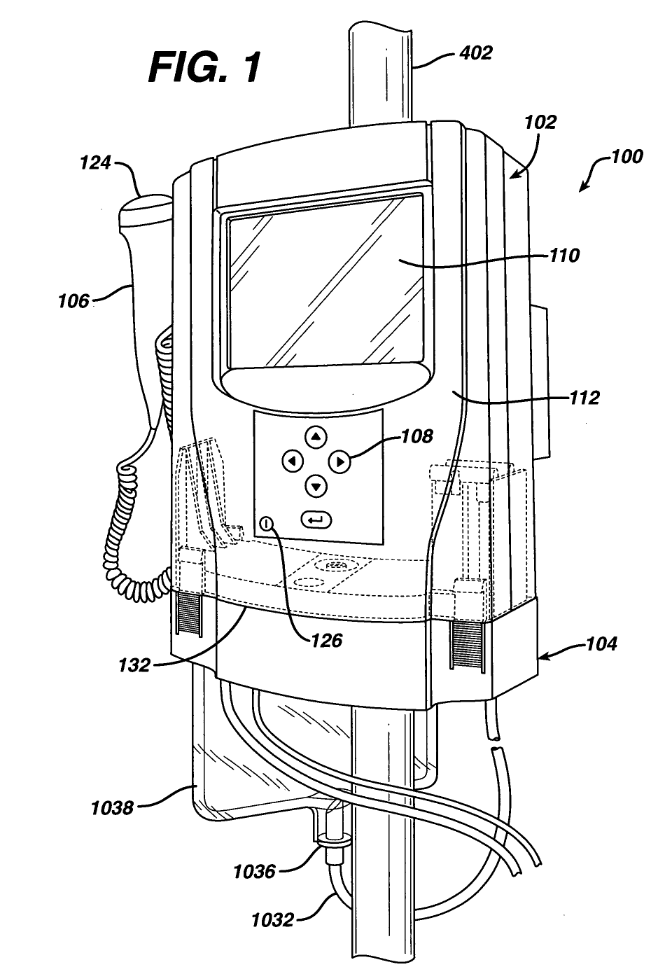

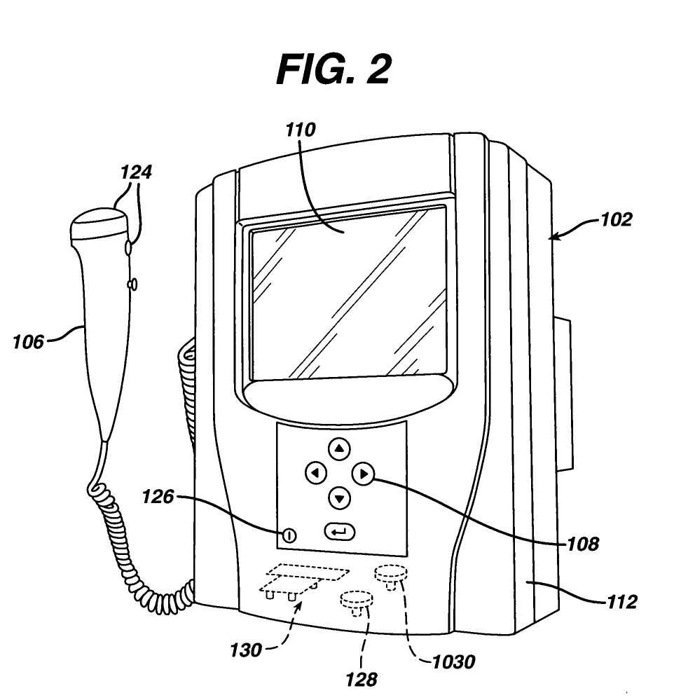

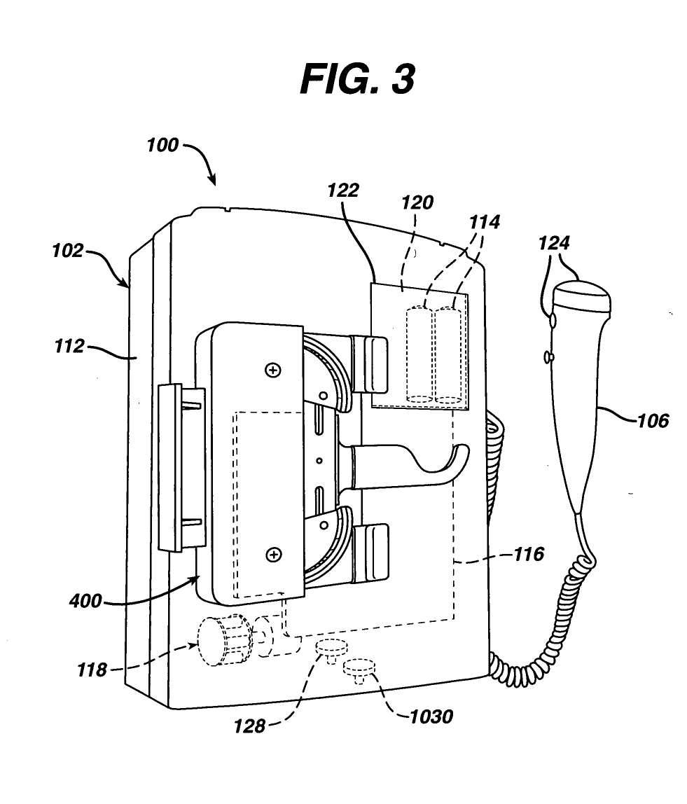

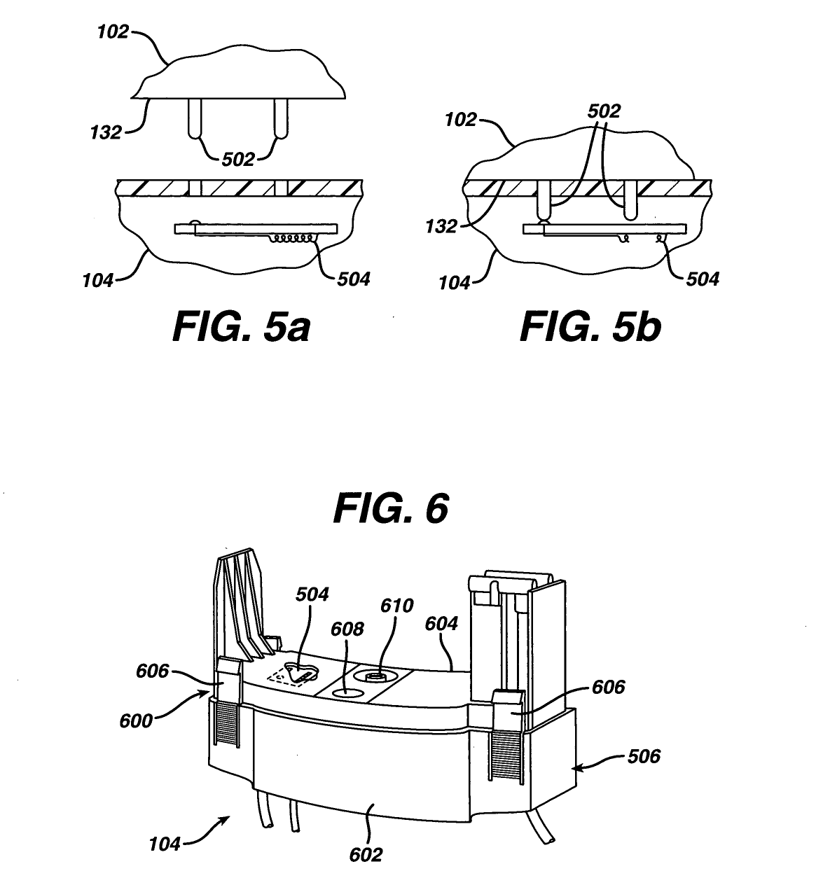

FIGS. 1 through 17 illustrate generally a system that can be used to both assess urinary function and to perform endometrial ablation. Referring now to FIGS. 1 and 2, the system 100 includes a control device 102 that controls operation of the system, at least one module 104 that can be removably coupled to the control device, at least one input device, such as the illustrated input pendant 106 and/or keypad 108, and at least one output device, such as the illustrated display screen 110. As will be described in more detail below, the control device 102 is designed to be removably coupled to any one of a plurality of modules 104 at any given time, including at least one that will support a test to assess incontinence, and at least one that will support endometrial ablation. As each module is uniquely suited to support a different type of diagnostic test or medical procedure, the resulting diagnostic system is not only readily portable, but is also extremely versatile in that the single control device, in conjunction with a plurality of small modules, is capable of performing an array of diagnostic tests or other procedures.

The control device 102 includes a housing 112 for housing various components, including one or more batteries 114, an electronics assembly 116, a pump device 118 including a motor, and various other circuitry. Batteries supply power to the control device 102, and are contained within a battery compartment 120 that is accessible by removing the battery cover 122 that forms part of the housing 112. The control device further includes an input keypad 108 for allowing a user to input data (such as patient name or other identifier, numeric identifiers, patient history, date etc.) and an input pendant 106 including one or more switches 124 that allow user input of additional information (i.e., event input based on patient feedback), and an activation switch 126 for turning the device on and off. The pump device 118 and at least one pressure transducer 128 are also contained within the housing. The pump device is electrically coupled to the battery and the electronics assembly, and the pressure transducer is electrically coupled to the electronics assembly. The control device 102 may also include a pole mounting mechanism 400 for mounting the control device on a pole 402. The device may also include an interface 130 including appropriate electrical pinouts to enable the control device to communicate for purposes of battery recharging or printing of patient test data, or for communicating with an endometrial ablation module as will be described further below.

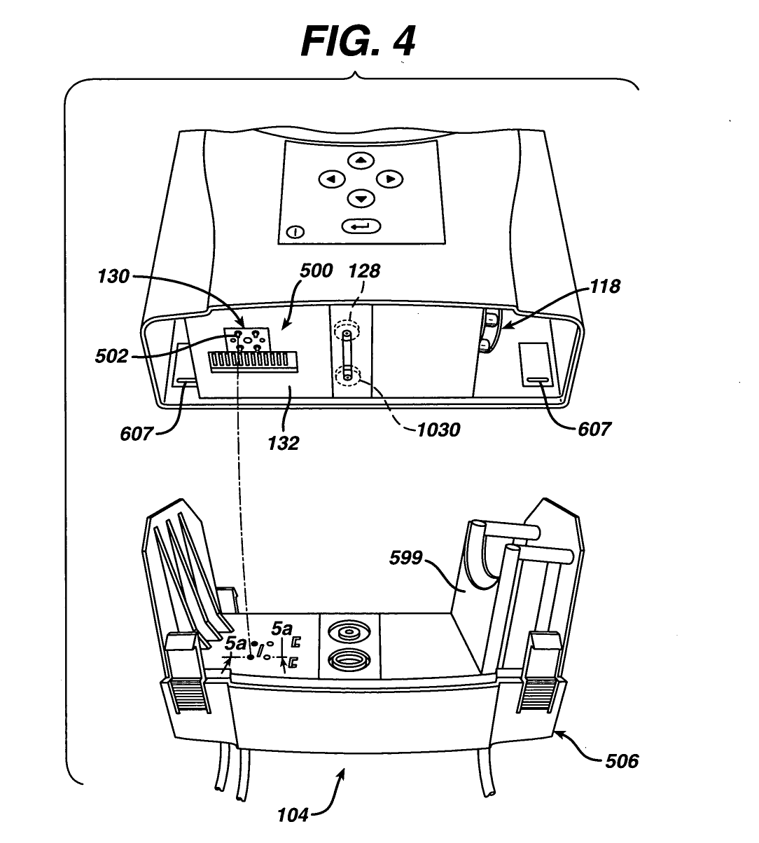

As indicated above, any one of a plurality of modules 104 can be removably coupled to the control device 102, and the control device is designed to uniquely identify the attached module and perform routines specific to that module. Thus, the control device includes a module detection mechanism 500 capable of identifying the attached module that is electrically coupled to the electronics assembly (see FIG. 4). This module detection mechanism includes one or more identification probes 502 that project from the interface side 132 of the control device and are electrically coupled to the electronics assembly. The modules 104 may include one or more apertures in the module housing 506 that are designed to receive therein the identification probes when the module is removably coupled to the control device. When so coupled, the identification probes will bridge one or more module identification elements or components 504, such as resistors, capacitors, fuses or other suitable electronic components, present within the module. The identification probes are electrically coupled to the electronics assembly 116 (described more fully below), which determines a value, such as resistance, associated with the module identification element(s) that they bridge. Each module is designed to have a value so that identification of this value by the electronics control assembly enables the control device to uniquely identify the attached module. In a preferred embodiment, the control device may include one or more sets of identification probes 502 at different locations, and different modules have module identification components 504 at different locations. The location, as detected by the control device, identifies the attached module. In yet another embodiment, the module identification component(s) may be coupled to an exterior side of the module housing so that apertures in the module housing are not required.

The module further includes at least one coupling element 600 for removably coupling the module to the control unit (see FIG. 6). In the illustrated embodiment, the module includes four coupling elements placed toward the ends of each of the front and rear faces 602, 604 of the test module. Each coupling element contains a tab element 606 that engages a corresponding ridge 607 (best seen in FIG. 4) on an interior surface of the control device when the module is removably coupled to the control device. To couple the module to the control unit, the coupling elements are depressed slightly. The module is then aligned with the control device as shown in FIG. 1, and the coupling elements released to allow engagement with the corresponding ridges described above. The module can subsequently be removed from the control unit by once again depressing the coupling elements and removing the module from the control device.

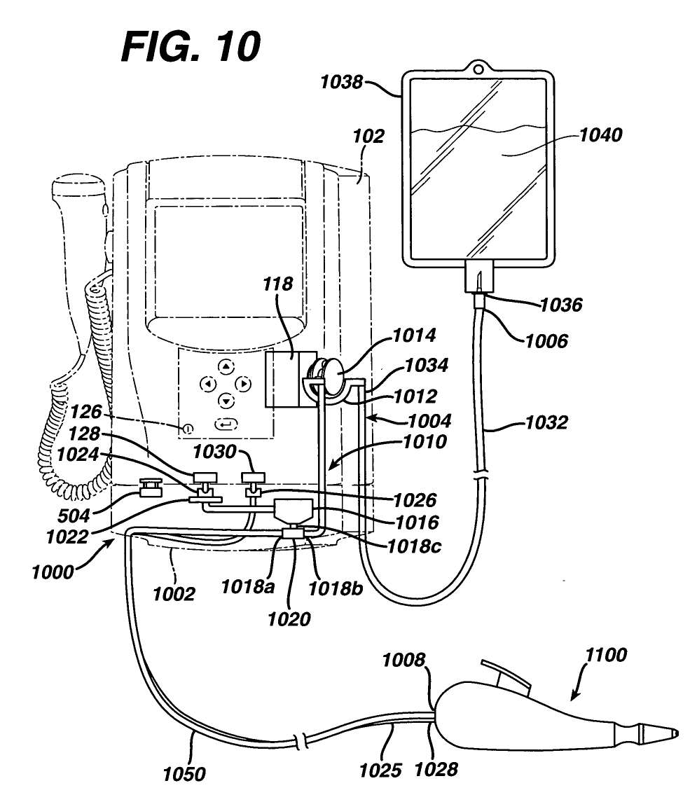

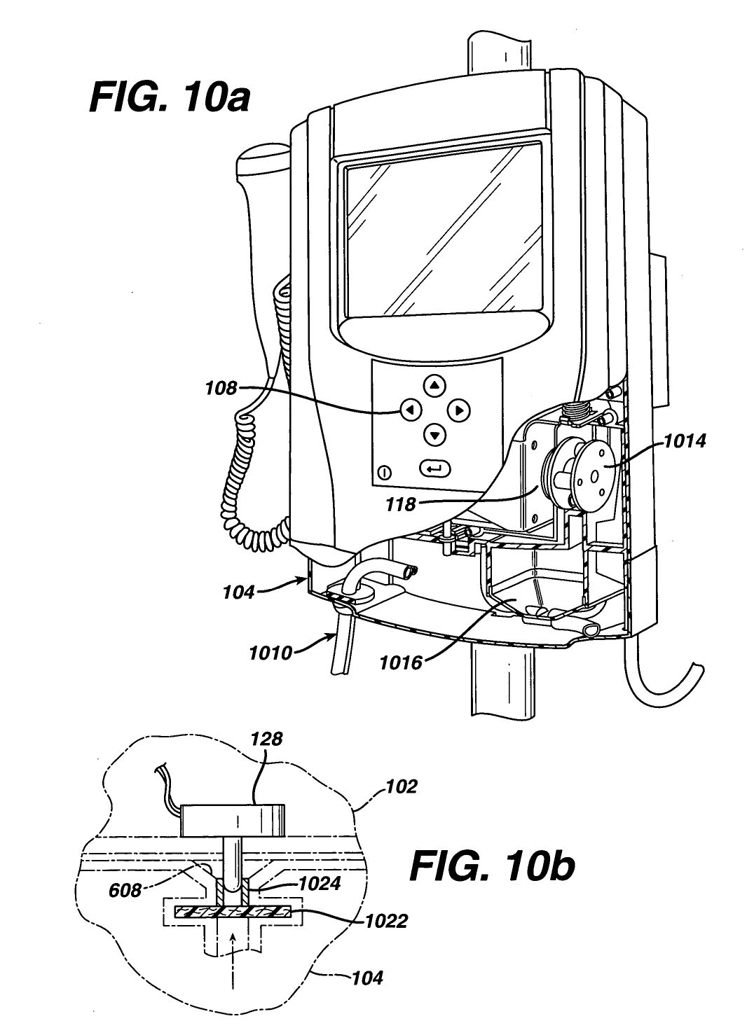

Finally, the module housing 506 includes first 608 and possibly second 610 ports therein as shown in FIG. 6. Each of the first and second ports are configured so as to define a recess capable of receiving a control device pressure sensor, such as a pressure transducer, therein when the module is coupled to the control device. For example, a first control device pressure transducer 128 is received within the first port recess 608 and comes in physical contact with a pressure interface 1024 (see FIG. 10) so that pressure changes at the pressure interface can be transmitted to and detected by pressure transducer 128 and converted to electrical signals that are sent to the electronics assembly for interpretation. Similarly, the second port 610 also defines a recess capable of receiving therein a second control device pressure transducer 1030. The first and second ports are further configured to form an airtight seal with the control device when coupled thereto, preferably by incorporating sealing elements such as gaskets or the like. Individual modules and their operation in conjunction with the control device will be described in greater detail below.

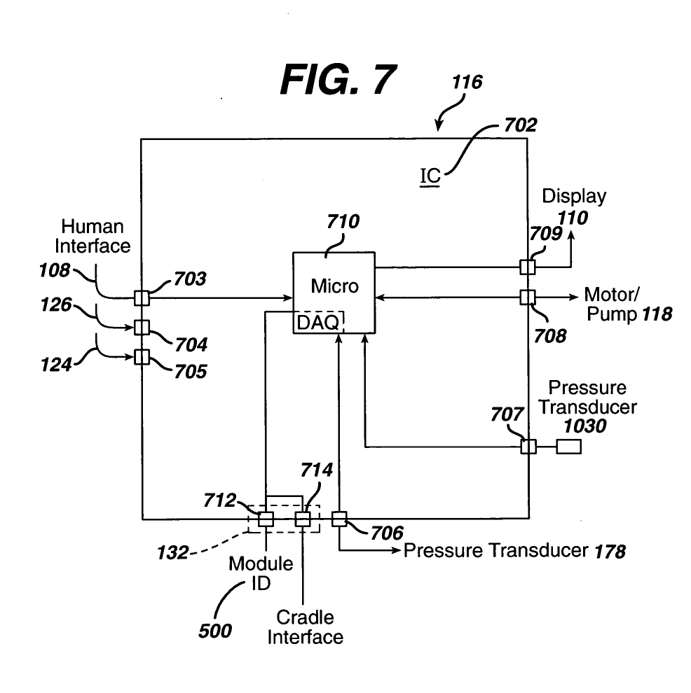

As indicated above, contained within the housing 112 of the control device 102 is an electronics assembly 116 (see FIG. 7) that is designed to control operation of the pump device 118, to acquire and format data from the pressure transducer(s), to drive a display 110 and/or other output device, and to accept and interpret input data, such as from switches 108, 126, and/or 124. The electronics assembly 116 consists of an integrated circuit board 702, hardware interfaces to the pump device 708, pressure transducer 706, 707, display 709 and switches 703, 704 and 705; and a microprocessor 710. The microprocessor 710 serves as the main controller for the diagnostic system and is supported by the custom integrated circuit 702 and powered by the batteries. Also included are interface connection elements including an electronic module identification connection 712 to the electronic detection mechanism 500, and electronic connections 714 that enable downloading of data to a printer or other external device.

The microprocessor 710 is programmed with a custom program file. In the illustrated embodiment, this software has multiple functions. First is the acquisition of input from the operator. This input data is captured from the input keypad 108, and/or switches 124, 126, pressure transducer(s) or other input device, depending upon which test module is in use. The software also controls operation of the pump device 118. Input data is interpreted and appropriate signals are sent to the pump device motor via the integrated circuit board 702. Yet another function is to acquire and condition data from the pressure transducer(s). This data is then sent in the appropriate format to the display 110, along with applicable pump device data in the form of volume or time information. Finally, as indicated above, the software receives input from the module detection mechanism 500 and interprets this input to determine which test module is coupled to the control device.

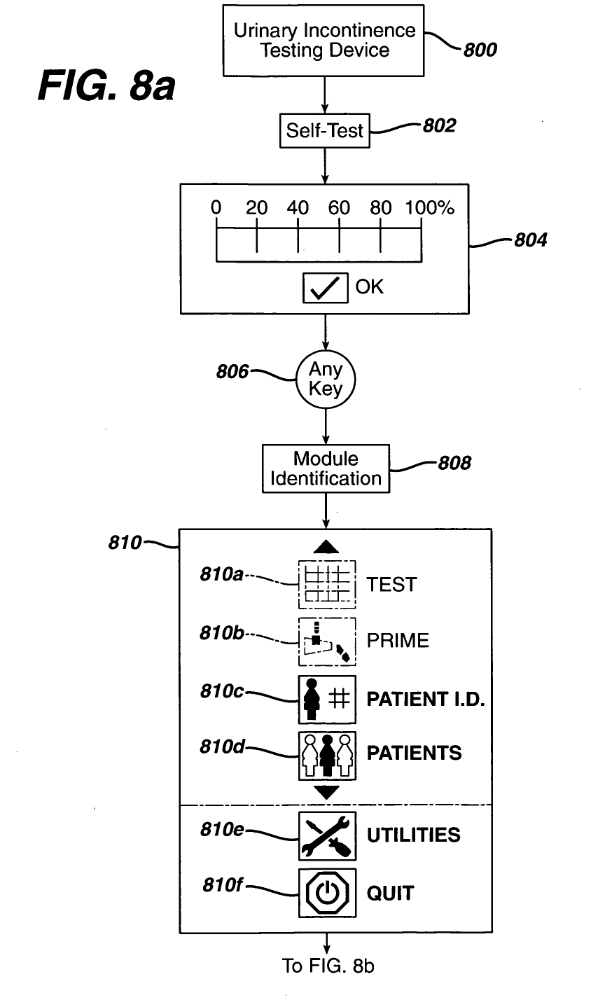

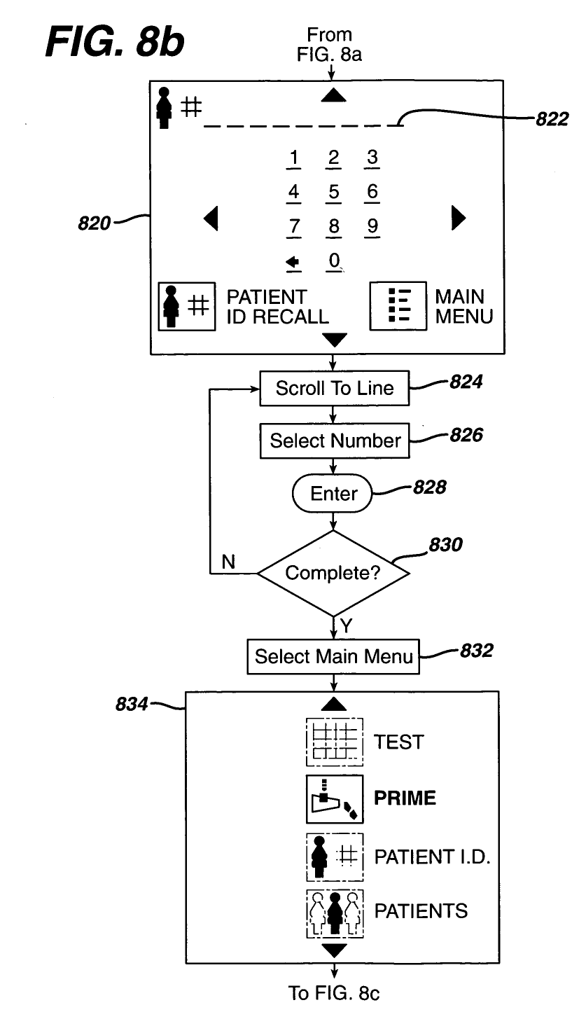

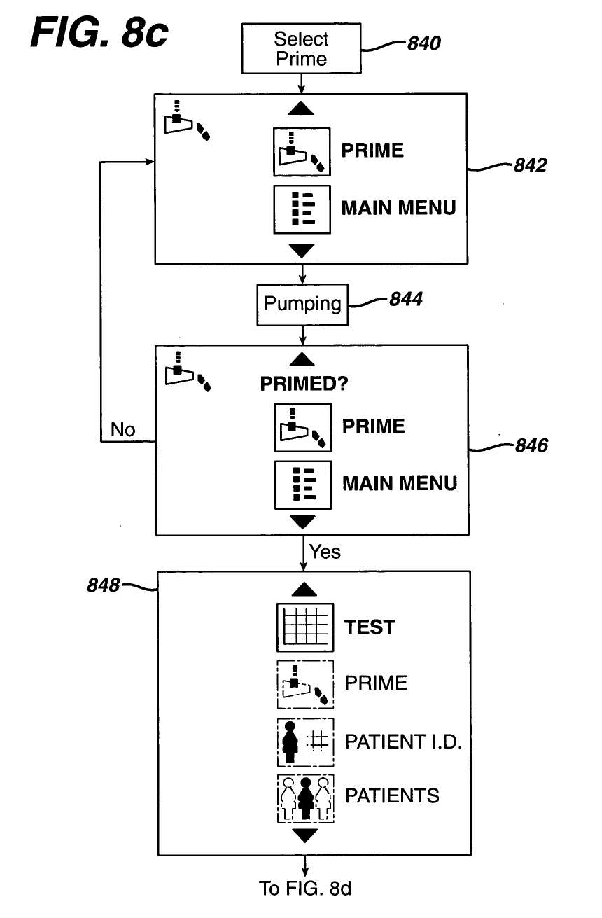

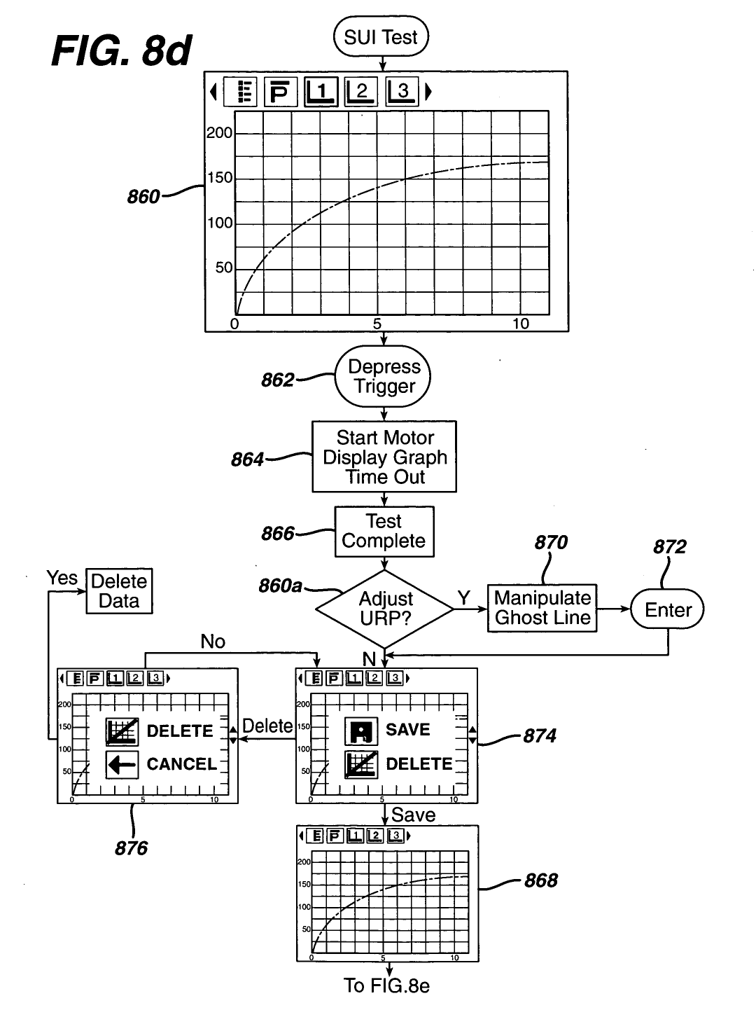

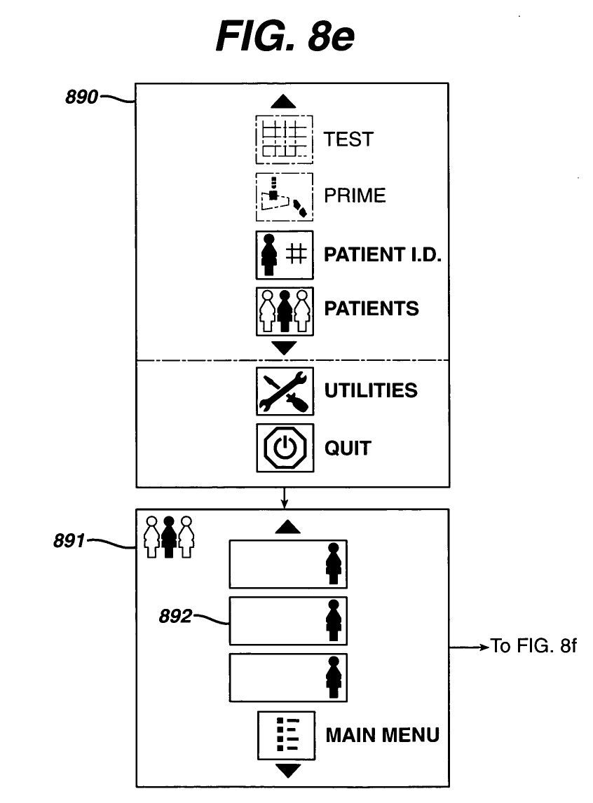

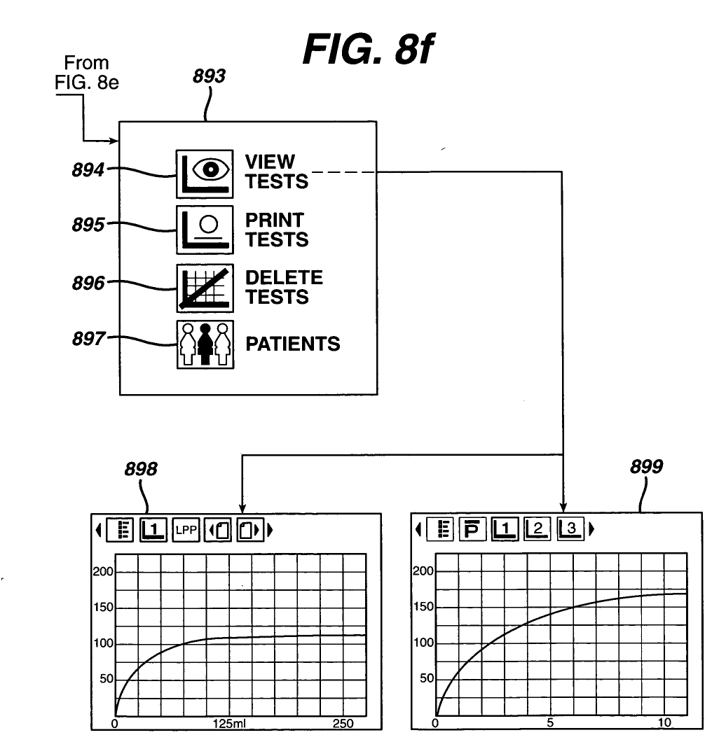

FIGS. 8a-8f are flow diagrams illustrating operation of the diagnostic system software and features of the system graphical use interface for a preferred embodiment of the invention. When the system is powered on, the user is first presented with a welcome screen. While this screen is being displayed the system is undergoing a self-test routine 802 to test the integrity of system hardware and software components. Upon completion of this routine, the user is provided with information relating to the amount of available system memory 804. Following the pressing of any key 806 on input device 108 by the user, the system identifies the attached module 808 as described above, and following such identification, the processor executes a software subroutine specific to the identified module. For each software subroutine, however, a main menu is displayed next, such as that indicated by reference numeral 810. In the illustrated embodiment, the main menu includes six possible selections. “Utilities” enables the user to access various system features, such as setting the date, time etc, or adjusting the brightness or contrast of the screen; “Quit” terminates the session; “Patients” enables the user to access any previously stored data relating to other patients and tests already performed; “Prime” initiates the pump priming process; “Patient ID” enables the user to enter a patient identification number; and “Test” initiates a software subroutine specific to the attached module to carry out the desired test procedure. In the presently described embodiment, the software and user interface associated with the “Prime,” “Utilities,” “Quit,” and “Patient ID” selections are substantially the same for each software subroutine. The “Test” and “Patients” selections, however, are different for each test module. Each of these selections will be described in greater detail below.

As is illustrated in FIG. 8a, the first time the main menu is displayed both “Test” and “Prime” appear in a different color or shade from the other options, indicating that they are not currently available. This is to ensure that patient identification information is entered before proceeding with any priming or medical procedures. The user may select the “Patient ID” option by scrolling using the appropriate arrows on the input keypad 108. Following this selection the Patient ID screens appears 820 (FIG. 8b). In the illustrated embodiment, the patient ID consists of a nine digit integer. To enter the patient ID, the user scrolls to a selected blank using the left and right arrows and/or left and right arrows on the input keypad 108 (824) to select desired numbers. Once the desired number is selected, the user presses ENTER; the selected number will then appear in the rightmost blank. Subsequent numbers are selected as described above, and will appear in the rightmost blank while previously selected numbers move to the left. This process is completed until all blanks are filled in. In one embodiment, there is a default value for each blank, such as 0, and the user may proceed with testing by accepting the default patient ID number consisting of all 0's. Once complete patient identification information is entered, the user selects the “Main Menu” option 832, which returns to the main menu screen. At this point, however, the “Prime” option become available 834 (and “Patient ID” is no longer available).

Before performing any procedure that requires fluid to be infused into the patient or into a device, priming operations must be performed to ensure that the fluid infusion lines (tubing) are filled with fluid and not air. Referring now to FIG. 8c, the user selects the “Prime” option 840 by using the arrow keys to select the option, and then pressing the enter key. The Prime screen then appears. According to one embodiment, the Prime screen includes two options as indicated at 842: “Prime” or “Main Menu.” In another embodiment, the Prime screen is particular to each module, and may present only one option to initiate priming. Selecting the Prime option causes the pump to start and run for a predetermined amount of time, such as 20 seconds, and then automatically shuts off. The user is then presented with a screen 846 at which the user can accept the prime as complete (MAIN), or choose to reprime (PRIME). When priming is accepted as complete, the main menu once again appears, this time with “Test” as an option 848. In another embodiment, priming operations may be specifically tailored for different test modules. For example, as will be described in more detail below, the SUI test modules includes a hand actuator including an activation button 1118 or 1128. The system may be designed so that following display of the Prime screen, pump priming operations can be initiated by depressing the activation button.

With priming complete, the testing or other medical procedure can begin. As indicated above, the enabled procedure depends on the attached module, and accordingly, the software and graphical user interfaces relating to each module will be discussed in greater detail below in conjunction with the detailed description of each module.

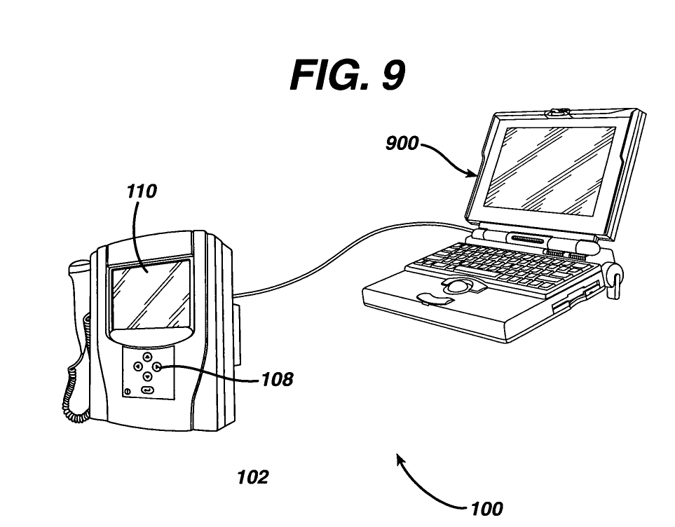

In an alternative embodiment of the invention illustrated in FIG. 9, the control device 102 is electrically coupled to a laptop/standard computer 900, and the microprocessor and associated software reside in the computer.

As indicated above, the diagnostic system described herein has particular application to urodynamics in that it enables clinicians to diagnose a plurality of urinary incontinence problems when used with specifically designed testing modules (to be discussed hereinafter). As a miniaturized urodynamic tool, the control device 102 in conjunction with modules 104 can measure urethral resistance pressure (URP), voiding flow (Uroflometry), and bladder dysfunction (Cystometrogram (CMG)). The URP module will be described further below. Details regarding other modules to assess incontinence can be found in U.S. Application No. 2003/0023135, which is incorporated herein by reference in its entirety.

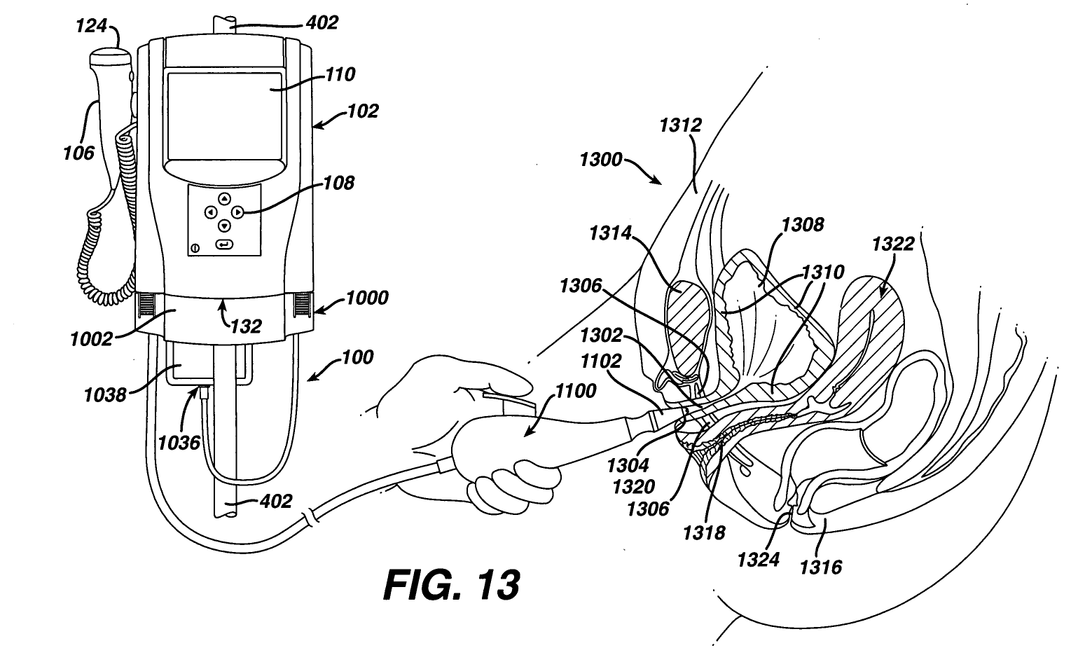

Before proceeding with a discussion of individual test modules, to assist the reader a brief overview of the female urinary system will be described with reference to FIG. 13. The female urinary system 1300 includes an elongated urethral canal 1302 having a urethral meatus (entrance) 1304 and having a substantially circular-shaped urethral sphincter muscle 1306 attached thereto, and a bladder cavity 1308 surrounded by a detrusor muscle 1310. The detrusor muscle 1310 also surrounds and supports the urethral canal 1302. The bladder cavity 1308 is in close proximity to the abdominal wall 1312, the pubis bone 1314, the pelvic floor 1316 (levator ani muscle), the vaginal canal 1318, the clitoris 1320, the uterus 1322 and the anal sphincter muscle 1324.

FIGS. 10-13 illustrate one embodiment of a stress urinary incontinence testing module (SUI) 1000 for diagnosing the involuntary loss of urine during physical activities such as coughing, sneezing, laughing or lifting. The SUI testing module 1000 includes a SUI module housing 1002 that can be removably coupled with the control device 102 as described above. The module housing may be in the form of a plastic disposable cartridge. Within the module housing is a tubing assembly 1004 including a fluid inlet 1006, a fluid outlet 1008, and a first fluid conduit 1010 extending therebetween. Tubing loop 1012 forms part of the tubing assembly and is positioned so that, when the SUI testing module is coupled to the control unit, the stator 1014 of the pump device 118 in the control unit 102 cooperates physically with the tubing loop 1012 so that the pump device operates as a peristaltic pump to pump fluid through the first fluid conduit 1010. To assist in this regard, a tubing guide 599 aids in positioning a portion of the tubing assembly so that it will properly and effectively engage the peristaltic pump. According to the illustrated embodiment, tubing guide 599 has a substantially U-shaped configuration, however, many other configurations are suitable, as the principles of operation of peristaltic pumps are well known in the art. Tubing member 1050 also forms part of the first fluid conduit. The module housing 1002 also includes a pressure chamber 1016 for dampening pressure fluctuations that may be caused by operation of the pump device. The pressure chamber 1016 is in fluid communication with the first fluid conduit 1010 via valve openings 1018a-c of three-way valve member 1020. The pressure chamber is filled primarily with air, but varying amounts of fluid may also be present. Positioned at a distal end of pressure chamber 1016 is a filter component 1022 designed to isolate fluid from electronic elements of the system 100. In this regard, filter 1022 may be a hydrophobic filter that allows air to pass into pressure interface 1024, but not liquid. When the testing module is coupled to the control device 102, pressure interface 1024 is in physical contact with pressure transducer 128 of the control device so that pressure fluctuations within the pressure chamber 1016 and pressure interface 1024 can be transmitted to and sensed by the pressure transducer, and subsequently transmitted to the electronics assembly as indicated above. In this manner, the control device measures pressure within the first fluid conduit of the tubing assembly of the SUI testing module, which substantially corresponds to the pressure within the urethral canal as described more fully below.

The SUI testing module 1000 tubing assembly also includes a second tubing member 1025 having a channel therethrough forming a second fluid conduit between a proximal end 1026 and a distal end 1028.

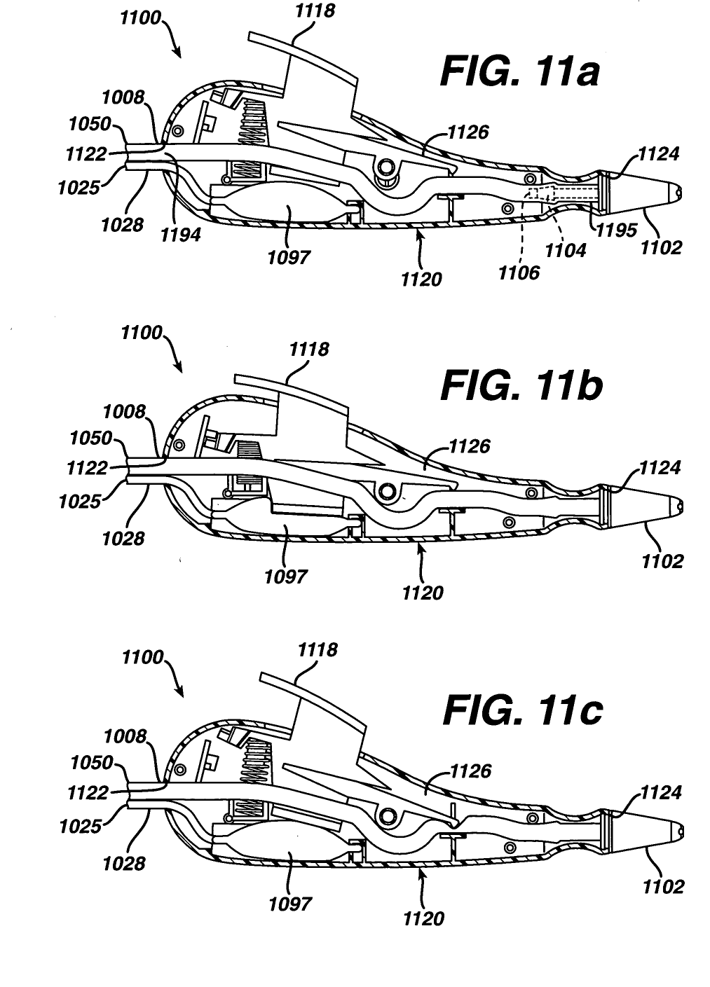

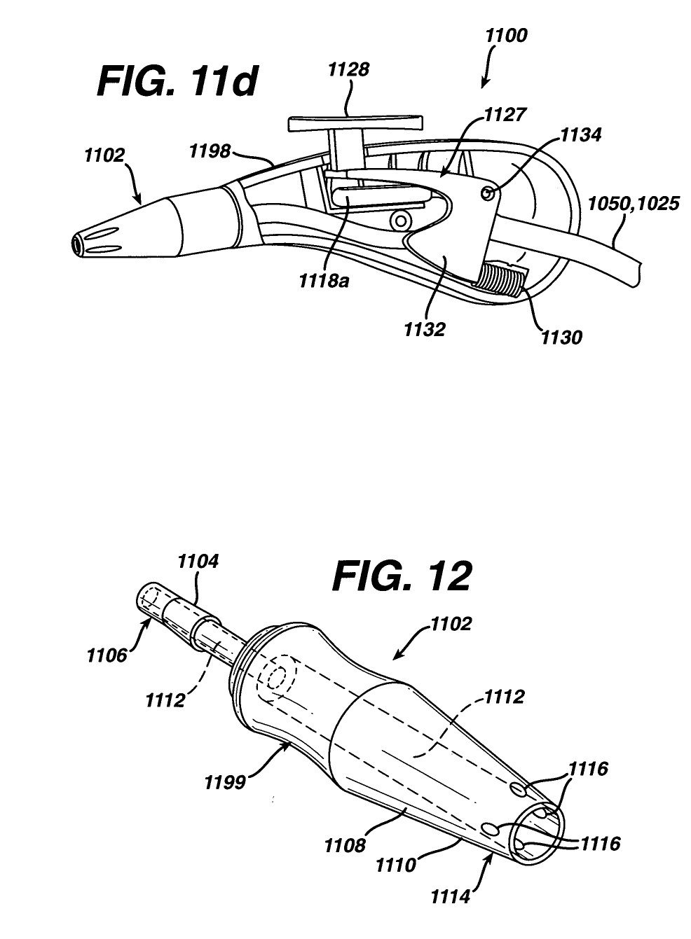

Referring now to FIGS. 11a-c, the SUI testing module may also include a hand actuator 1100 having an insert device such as a meatus plug device 1102 attached thereto. The meatus plug device 1102 (see FIG. 12) includes an attachment member 1104 at a proximal end 1106 coupled to a plug or insert element or member 1108 at a distal end 1110, and a channel 1112 extending therethrough allowing fluid flowing through the first fluid conduit to flow through the meatus plug device. The distal end 1114 of the plug element may also include one or more transversely aligned apertures or openings 1116 therein approximately equally spaced apart from one another around the exterior surface of the distal end. As the outer diameter of the distal end at the location of the apertures is less than the diameter of the inner wall of the urethral canal at that location (described more fully below), one or more of the apertures 1116 can be used for assurance of fluid flow into the urethra during actual operation.

In one embodiment, the hand actuator further includes a hand-sized housing or casing 1102 including therein an initiator element 1118 (FIGS. 11a-c) that is in fluid communication with tubing member 1025. Preferably, initiator element is an air bladder 1097 coupled to a distal end 1028 of the tubing member 1025. The proximal end 1026 of tubing member 1025 coupled to a pressure interface 1026a that is positioned so that, when the SUI testing module is coupled to the control device, pressure within tubing member 1025 can be sensed by pressure transducer 1030. As a closed system, pressure on the activation button 1118 can be sensed at the pressure interface 1026a by pressure transducer 1030, and interpreted by control device 102 as a signal to initiate and/or deactivate the test.

The hand actuator 1100 further includes a fluid conduit 1050 extending between an outlet 1195 and an inlet 1194 that is coupled to (integrally or otherwise) an external tubing conduit leading to a fluid source, such as the first fluid conduit 1010 of the SUI test module. Alternatively, the hand actuator may be designed to include therein the fluid source. The fluid outlet 1195 is in fluid communication with the insert member channel of the meatus plug device. An activation device 1127 including a trigger 1128 extends through an opening 1118a to an exterior of the casing. The activation device 1127 is movable between a first rest position (shown) and a second activated position. In the first position spring 1130 exerts force on coupling member 1132, causing it to pivot relative to pivot element 953 and pinch the distal ends of at least tubing member 1050 to prevent fluid flow therethrough. When in the second position, movement of the trigger causes the coupling member 1132 to pivot to a point at which it no longer pinches tubing member 1050. Further, trigger 1128 may also compresses air bladder 1097 to initiate testing as described above in connection with initiator element.

The plug element 1108 is configured so that, when inserted into the urethral meatus of a patient (see FIG. 13), it will substantially block or prevent fluid flow out of the urethra, as well as into the urethra other than through the meatus plug device channel 1112.

Further, when inserted, the plug element is positioned distal of the urethral sphincter 1306 (toward the outside of the body) as shown in FIG. 13. In the embodiment shown in FIG. 12, the distal end or distal portion 1114 of the plug element is substantially conical in shape, and decreases in diameter toward its distal end 1114. A proximal portion 1199 is configured to engage the inner wall of the urethral canal to substantially prevent fluid flow therebetween. Other shapes, however, are also possible so long as fluid flow into or out of the urethral is substantially blocked (other than through the meatus plug device channel) and the plug element remains located distal of the urethral sphincter. The meatus plug device 1102 is made of a biocompatible material, such as stainless steel or polypropylene. The meatus plug device may be disposable, but may also be made of a sterilizable material so that it can be reused.

The first fluid conduit 1010 of the tubing assembly also includes an elongated single lumen tubing member 1032 having a first end 1006 and a second end 1034 and a fluid channel extending therethrough. A spike device 1036 is coupled to the first end 1006 of the single lumen tubing member for attachment to a fluid bag 1038 (having a fluid 1010 therein) in a manner well known in the art. As described above, the meatus plug device and first fluid conduit are coupled to one another such that fluid from the fluid source traveling through the first fluid conduit may pass through the insert member (via the channel therein) and into the urethral canal distal of the urethral sphincter. Further, as the first pressure interface 1024 is in fluid communication with the first fluid conduit and ultimately the urethral canal, pressure at the pressure interface substantially corresponds to the pressure within the urethral canal distal of the urethral sphincter.

Use of the system 100 including a SUI testing module 1000 is as follows. First, the SUI testing module is removably coupled to the control device 102 in the manner described above. The physical coupling causes the identification probes 502 of the control unit to engage the module identification element(s) 504 of the SUI testing module, enabling the control device to identify the SUI testing module. The physical coupling also brings pressure interface 1024 in physical contact with pressure transducer 128 as described above so that pressure changes at the pressure interface can be detected by the pressure transducer and transmitted to the electronics assembly for interpretation. The pressure interface 1026a at the proximal end of tubing member 1025 similarly comes in contact with pressure transducer 1030 so that pressure within tubing member 1025 can also be detected. Finally, the tubing loop 1012 is brought into physical contact with the pump device 118 so that the pump device can drive fluid through the first fluid conduit by peristaltic motion, as described above.

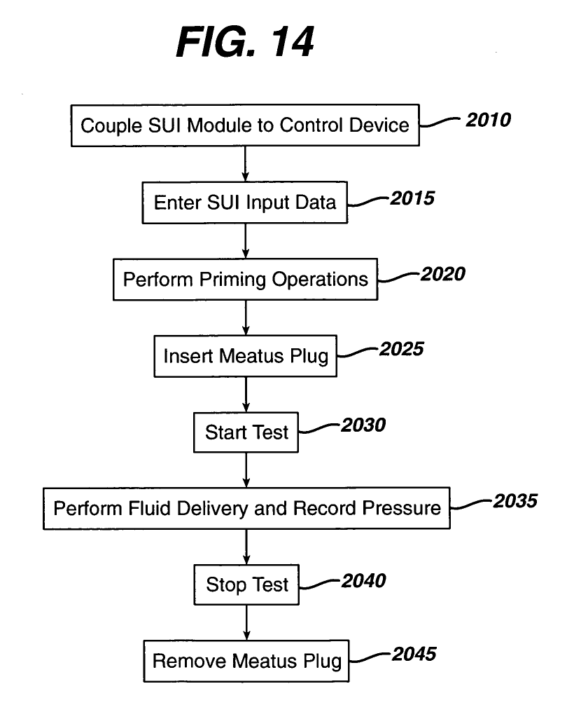

As shown in FIG. 14, once the SUI testing module 1000 is coupled to the control device 102 (2010), the operator enters appropriate input data into the keypad 108 or other input device (2015) for the SUI test (described in more detail below). This data is received and interpreted by the microprocessor 710 and applicable information is sent by the microprocessor to the display 110. Priming operations are then performed (2020) to ensure that the first fluid conduit 1010 contains fluid. At this point, the microprocessor is ready to start the test routine.

The meatus plug 1102 is inserted into the meatus of the urethra (2025) and the test is started (2030) by pressing the activation button as described above. This in turn sends instructions to the pump device via the integrated circuit. The pump device then pumps fluid 1040 through the first fluid conduit 1010 and meatus plug device channel 1112 and into the urethral canal distal of the urethral sphincter (2035). As fluid pressure builds in the urethral canal 1302, pressure in the pressure chamber 1016 also builds. This pressure is transmitted through the filter component 1022 and pressure interface 1024 to the pressure transducer 128, which receives the pressure data and transcribes it into an electrical signal. The electrical signal from the pressure transducer is sent to the microprocessor 710 via the integrated circuit 702 where it is acquired and conditioned. The information is then sent to the display 110 via the integrated circuit. The microprocessor ends the test after a specified amount of time, or upon receipt of input from the user by sending an “off” signal to the pump motor drive. Once the test has been completed, the operator disengages the activation button 1118 (step 2040) and removes the meatus plug element from the meatus 1304 (2045).

Referring once again to FIGS. 8a-f, and in particular FIG. 8d, when the “Test” option is selected the SUI test can be performed. The SUI Test screen appears 860, and the user initiates the test by depressing the trigger 1128 or movable shell 1126 (862) to allow fluid flow into the urethral canal as described above. The motor is then activated and the pump device pumps fluid into the urethral canal for a predetermined period of time, preferably 15 to 20 seconds. During this time a graph (see 860) is continuously displayed illustrating measured pressure on the vertical axis (preferably in cm of water) versus time on the horizontal axis. As fluid is pumped into the urethral canal, pressure within the urethral canal distal of the sphincter continues to increase until that point in time at which the urethral sphincter yields (open) under the force of the pressure within the urethral canal. At that point the pressure curve becomes substantially flat, as illustrated in FIG. 8d, since the sphincter is open and fluid is filling the bladder. The value of the flat portion of the curve is considered the “urethral resistance pressure (URP),” and can be obtained from the displayed graph. On completion of the test (after expiration of the predetermined time period the pump device stops), the graph remains, and the user is preferably provided with an option to adjust the software generated URP value (860a) before saving the test results. To adjust the URP value, the user uses the up and down arrows to manipulate a horizontal line which indicates the URP value that appears on the screen (870). When the ghost line is at the desired value, the user presses enter (872).

Once the final URP value is displayed, a Save/Delete screen 874 is overlayed on the screen. If the user selects the “Save” option, the test results are saved in memory. If the user selects “Delete” from the Save/Delete screen 874, the user is then presented with the Save Test screen 876. If “Delete” is chosen the test is deleted, but if “Cancel” is selected, the user is returned to the Save/Delete screen.

According to one embodiment, test results for up to three out of six possible tests may be stored. Once three tests have been stored or six tests have been run, whichever comes first, the control unit 102 will disable the module identification component 504 via the identification probes 502. After testing is complete, the user may return to the main menu by selecting the “Menu” option from the Test Complete screen.

One option available from the Main Menu, as stated above, it “Patients,” which allows the user to access patient and test data previously stored. According to one embodiment illustrated in FIG. 8e, when “Patients” is selected from the Main Menu, a Patients Screen 891 appears. On this screen, options for each patient and test for which data has been stored 892 are presented and selection of one of these options causes a Patient Test Menu 893 to be displayed (FIG. 8f). Selecting “Delete” 896 will present the user with the option to delete the stored data for that patient/test, and selecting “Print” 895 will enable the user to print the stored data. The Print option will only be available (will not be greyed out) when the control device is coupled to a cradle, or otherwise appropriately coupled to a printer. Selecting “View Test” will cause a Patients Test screen 898 or 899 to appear depending on whether stored data is a CMG (898) or a SUI (899) data set. The Patients Test screen may vary depending on the test module that is attached. For example, for the SUI stored data, the Patients Test screen is the screen illustrated by 899, whereas for the CMG data (discussed below), the Patients Test screen is the screen illustrated by 898. The Patients Test screens provide the user with the option to view data relevant to the particular form of test performed.

As indicated above, the results obtained from the SUI test is the urethral resistance pressure (URP), which is the back-pressure necessary to force open the urethral sphincter muscle 1306 from the reverse or opposite direction from which fluid normally flows. A major advantage of the SUI testing module 1000 is that the insert or plug element 1108 of the meatus plug device 1102 only enters the external urethral canal (meatus) and does not cause any discomfort associated with passing a catheter through the internal urethral sphincter. Thus, the diagnostic system disclosed herein having a SUI module 1000 is less invasive and more comfortable for patients. Further, the testing procedure for the SUI module 1000 is easy to implement, quick to perform, and does not require advance training by the clinician and/or physician.

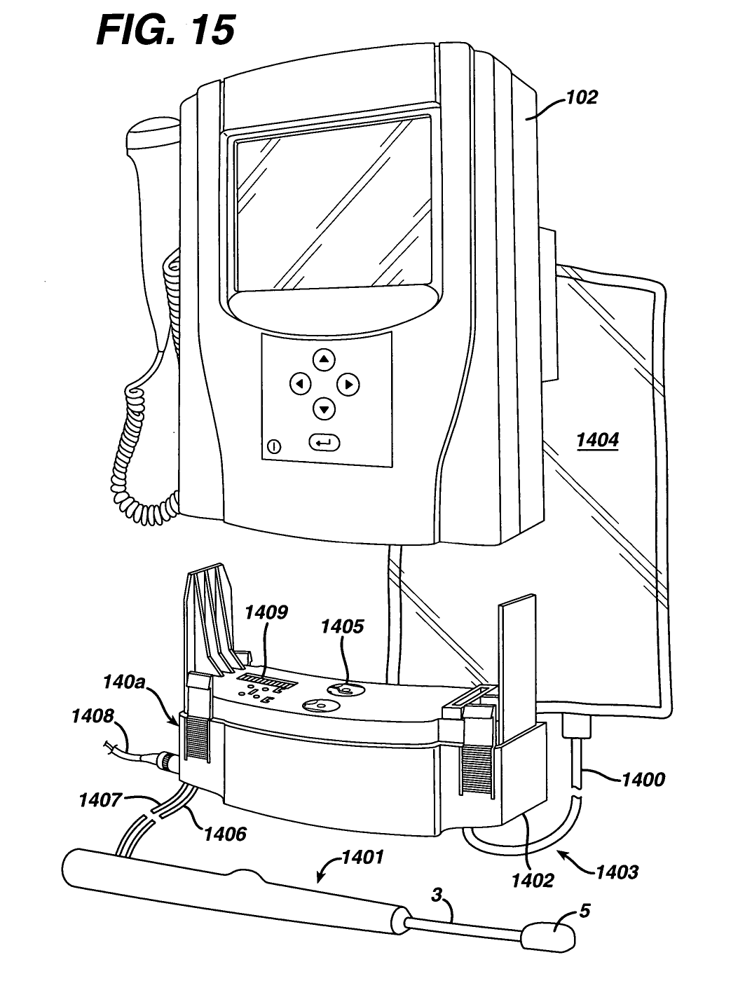

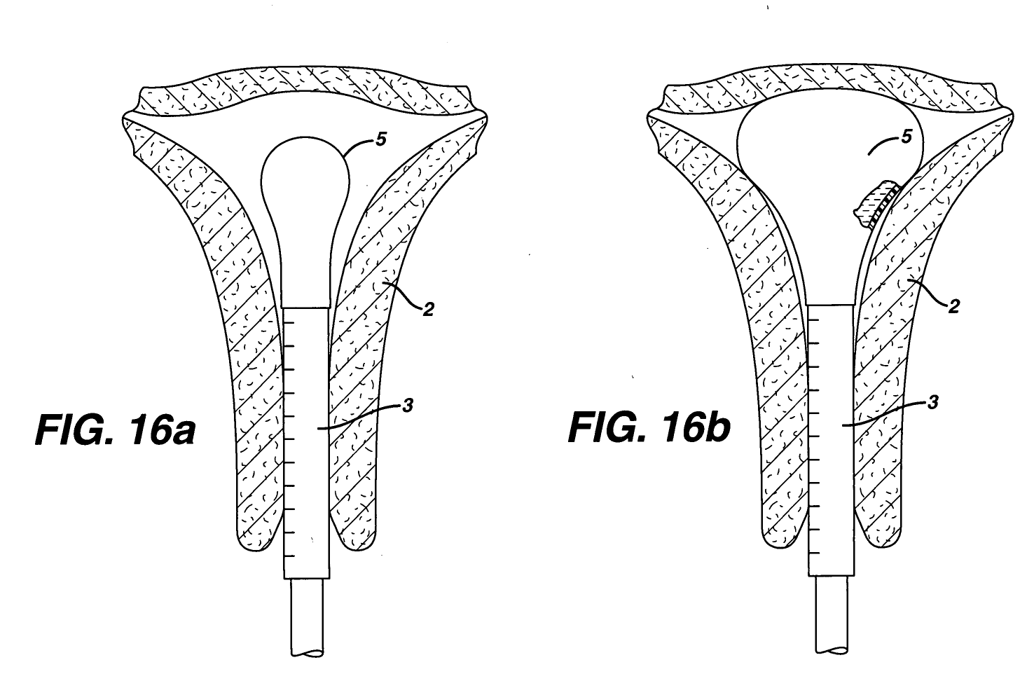

The control device described above can also be used in conjunction with a different module that supports use of the system to perform endometrial ablation. This module will be identified by the control device in a manner similar to that described above. As indicated previously, one known system for performing endometrial ablation is called the Thermachoice® system, which is manufactured and sold by Gynecare, a division of Ethicon, Inc. of Somerville, N.J. This system is described in. greater detail in U.S. Pat. Nos. 4,949,718, 5,105,808 and 5,704,934, which are incorporated herein by reference in their entirety. As shown in FIGS. 15 and 16a and 16b, the endometrial ablation system 1401 generally includes an inflatable distendable bladder 5 that is attached to rigid tubing 3 such as a catheter. The distendable bladder is inserted within the uterus 2 in a deflated configuration as shown in FIG. 16a, and is subsequently filled with a fluid so as to assume the inflated configuration shown in FIG. 16b that substantially conforms to the shape of the interior of the uterus. The fluid is then heated to a temperature of preferably about 87 degrees Celsius for a period of about 8 minutes so that the heated fluid effects necrosis on the surrounding endometrial lining due to the bladder being in direct contact therewith. The pressure sufficient to ensure adequate contact with endometrial tissue to be necrosed should be maintained at about 40-240 mm Hg, and preferably be about 75 mm Hg.

The infusion of fluid into the distendable bladder, the pressure therein, and the heating of the fluid can be controlled by the control unit described above. Referring now to FIG. 15, the endometrial ablation system 1401 can be coupled to and controlled by the control unit 102 via another module 104a that supports endometrial ablation (the “GEA module”). The GEA module couples to the control device in the manner described above, and includes a tubing system 1403 that, as shown in FIG. 15, includes a fluid tubing inlet 1400 that extends from the bottom side 1402 of the GEA module and connects to a fluid reservoir 1404, such as a saline bag, that is preferably elevated to create hydrostatic pressure within the fluid tubing. A fluid tubing outlet 1406 is in fluid communication with the fluid reservoir tubing inlet, and extends from the module to the system 1401 to provide fluid through the catheter 3 to the distendable bladder 5 to inflate it. Although not shown directly in FIG. 15, the tubing within the module that connects the fluid tubing inlet and the fluid tubing outlet is configured such that it couples with the peristaltic pump as described in detail above in conjunction with the SUI module. Thus, the operation of the peristaltic pump will pump fluid from the fluid reservoir into or out of the distendable bladder.

The portion of the tubing inside the module between the peristaltic pump and the fluid tubing outlet is also in fluid communication with at least one pressure interface 1405. The manner of coupling of the pressure interface with a corresponding pressure transducer in the control device may be similar to that described above in relation to the SUI module, and similarly provides the control device with information regarding the pressure within the outlet tubing and thus within the distendable bladder.

The GEA module also preferably includes an external power connector 1408 to supply the power required to perform endometrial ablation. External power is preferred over an internal battery within the control device because of the amount of power required, which is typically approximately 42 watts over an approximately 8 minute time period.

Finally, the GEA module preferably includes a umbilical connector 1407 that electrically couples the endometrial ablation system 1401 to the module 104a to allow the control device to control the heating and fluid agitation elements of the endometrial ablation system via a 12 pin Bourns connector electrical interface 1409 and switches (not shown) that reside within the module. In the Thermachoice® device, the heater resides within the distendable bladder and includes both a heater coil and a stirring element to stir the fluid within the distendable bladder to thereby maintain a more uniform fluid temperature. The control device also monitors and controls the pressure within the distendable bladder by acquiring pressure information via the pressure interface 1405, and controlling the forward or reverse operation of the peristaltic pump to thereby increase or decrease fluid volume within the bladder.

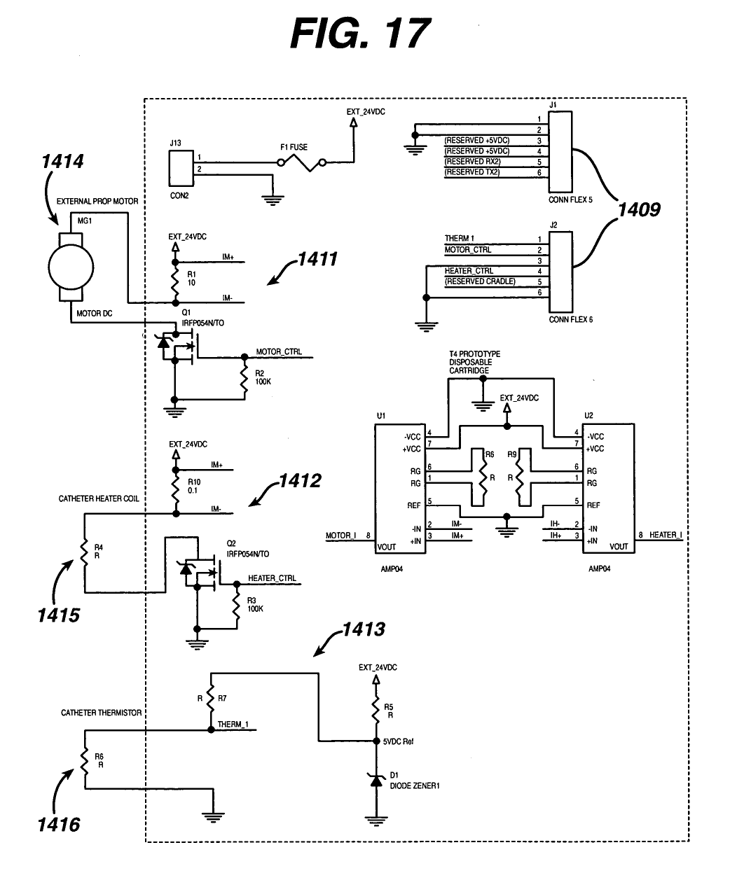

FIG. 17 illustrates the electronics within the GEA module in greater detail. The elements within the dotted line 1410 illustrate the circuitry within the GEA module, which include motor control circuitry 1411 for controlling the motor 1414 for the fluid agitator (if present) within the system 1401, heater control circuitry 1412 for controlling the heating element 1415 within the system, and temperature circuitry 1413 for obtaining temperature feedback from thermistor(s) 1416 or the like within the system 1401. This circuitry communicates with the control device via connector 1409 as shown. Although these electronic elements are shown within the GEA module, some or all could readily be incorporated into the control device to reduce the cost and complexity of the module, or could be incorporated into a reusable module rather than a disposable one.

The endometrial ablation procedure begins by connecting the GEA module to the control device, connecting the fluid inlet tubing 1400 to a source of fluid 1404 such as an IV bag, connecting the fluid outlet tubing 1406 and the umbilical connector 1407 to the endometrial ablation system 1401, and connecting the external power connector 1408 to a suitable source of power. The pump is then primed as described above, and fluid subsequently removed from the distendable bladder by reversing the direction of the pump. The distendable bladder is then inserted into the uterus, and the pump is operated in the forward direction to infuse fluid into the distendable bladder until a preset fluid pressure is reached, as is determined by the control device via monitoring the pressure interface. The pressure is then maintained while the fluid is heated preferably to a temperature of approximately 87 degrees Celsius and for a time period of approximately 8 minutes to accomplish the necessary ablation. As indicated above, the control device receives temperature feedback via electrical interface 1409 and controls operation of the heating and fluid agitation means of the endometrial ablation system 1401 by controlling internal on/off switches to these elements via the electrical interface. Thus, the temperature of fluid within the bladder can readably be maintained around a pre-determined set-point. The control device may also measure the volume of fluid infused into the bladder. This can be accomplished by recording the “on time” of the pump, and multiplying it by the rate of infusion, which is preferably approximately 1 ml/sec.

Thus, the system and modules described above enable a single portable system to readily and easily be used to perform both urinary assessment and endometrial ablation. Further, although the Thermachoice® device has been described in detail in relation to endometrial ablation, it is to be understood that any other known or developed device or system for performing endometrial ablation could be modified in a similar manner so as to be incorporated into a module for use in conjunction with the described control device. Accordingly, those skilled in the art will understand that many variations are possible without departing from the spirit and scope of the invention, which is limited only by the appended claims.

What is claimed is:

1. A medical system comprising:
a control device;
a plurality of modules each capable of being removably coupled to the control device;
wherein for at least a first one of the plurality of modules, when removably coupled to the control device, the medical system is capable of performing a test to assess urinary function, and wherein for at least a second one of the plurality of modules, when removably coupled to the control device, the medical system is capable of performing endometrial ablation.

2. The system according to claim 1, wherein the first and second modules each further comprise a tubing assembly forming a fluid conduit from a fluid inlet to a fluid outlet.

3. The system according to claim 2, wherein the control device further comprises a pump device, and wherein when the first or second module is coupled to the control device, the pump device engages the tubing assembly of the first or second module respectively for pumping fluid therethrough.

4. The system according to claim 3, wherein the pump device is a peristaltic pump.

5. The system according to claim 3, wherein the second module further comprises an endometrial ablation system including a catheter having a proximal end and a distal end, a distendable bladder attached to the proximal end for insertion into a patient's uterus, and a heater for heating fluid infused into the distendable bladder,
wherein the fluid inlet of the second module is capable of coupling with a fluid source and the fluid outlet is in fluid communication with an interior of the distendable bladder, and wherein when the second module tubing assembly is coupled with the pump, operation of the pump controls the flow of fluid from the fluid source into or out of the distendable bladder.

6. The system according to claim 5, wherein the control device further includes a pressure sensor, and wherein the second module further includes a pressure interface that, when the second module is coupled with the control device, is positioned relative to the control device pressure sensor so as to transmit pressure information thereto.

7. The system according to claim 6, wherein the pressure interface is in fluid communication with the tubing assembly of the second module at a position such that fluid pressure at the pressure interface substantially corresponds to pressure within the distendable bladder.

8. The system according to claim 7, wherein the second module further includes at least one temperature sensing element for sensing a temperature of fluid within the distendable bladder.

9. The system according to claim 8, wherein temperature information from the at least one temperature sensing element is provided to the control unit through an electrical interface, and wherein the heater is controlled by the control unit via the electrical interface.

10. The system according to claim 9, wherein the second module further includes an external power connector for coupling with a power source.

11. The system according to claim 5, wherein when the first module is removably coupled to the control device, the system measures Urethral Resistance Pressure to thereby assess urinary function.

12. The system according to claim 5, wherein the first module further comprises an insert member dimensioned for at least partial insertion into a patient's urinary tract and coupled to the first module fluid outlet so that fluid infused through the first module tubing assembly passes through the insert member and into the urinary tract.

13. The system according to claim 12, wherein the insert member is dimensioned for insertion into the urethral canal distal of the urethral sphincter, and the first module further comprises a pressure interface in fluid communication with the urethral canal distal of the urethral sphincter when the insert member is so inserted.

14. A medical system comprising:
a control device including at least one input device, at least one output device, a microprocessor, and a pump device;
first and second modules each individually capable of being removably coupled with the control device, wherein when the first module is so coupled the system is capable of performing a test to assess urinary function, and when the second module is so coupled the system is capable of performing endometrial ablation, wherein the first and second modules each including a tubing assembly extending from a fluid inlet to a fluid outlet, wherein when coupled to the control device, the tubing assembly is coupled to the pump device for pumping fluid therethrough,
the first module further including a device coupled thereto for infusing fluid into a patient's urinary tract, wherein feedback is provided regarding the infused fluid to the control device and the infusion of fluid into the patient's urinary tract is controlled by the control unit
the second module further including an ablation system coupled thereto for ablating an interior lining of the uterus, wherein feedback is provided from the ablation system to the control device and wherein the control device controls operation of the ablation system.

15. The system according to claim 14, wherein the ablation system further comprises a catheter having a distendable bladder attached to a proximal end thereof for insertion into a patient's uterus, wherein fluid is infused from the tubing assembly of the second module into the distendable bladder to inflate the bladder to approximate the interior of the uterus, and wherein the ablation device further includes a heating device for heating the fluid infused into the distendable bladder to thereby cause necrosis of the uterine lining.
